(19) United States
(12) Patent Application Publication
(10) Pub. No.: US 2020/0069207 A1
(43) Pub. Date: Mar. 5, 2020

(54) Neural Implant with Wireless Power Transfer

(71) Applicant: Philip R. Kennedy, Duluth, GA (US)

(72) Inventor: Philip R. Kennedy, Duluth, GA (US)

(73) Assignee: Neural Signals, Inc., Duluth, GA (US)

(21) Appl. No.: 16/675,635

(22) Filed: Nov. 6, 2019

Related U.S. Application Data
(63) Continuation-in-part of application No. 15/380,097, filed on Dec. 15, 2016.
(60) Provisional application No. 62/756,153, filed on Nov. 6, 2018.
(60) Provisional application No. 62/267,366, filed on Dec. 15, 2015.

(51) Int. Cl. A61B 5/0478; H02J 50/80; H02J 50/20; A61B 5/00

(52) U.S. Cl. CPC A61B 5/0478; H02J 50/80; H02J 50/20; A61B 2560/0214; A61B 5/0031; A61B 5/6868; A61B 5/686

(57) ABSTRACT

A neural implant system for communicating neural impulses generated by a brain of a patient having a body includes a neural implant electrode system that is configured to be implanted in a selected site of the patient's brain. A wireless power receiver and communication circuit is in communication with the neural implant electrode and is configured to be disposed within the patient's body at a predetermined location. An external telemetry and power unit is configured to provide power to and to communicate with the wireless power receiver and communication circuit. The wireless power source and transceiver circuit is configured to operate outside of the patient's body.

### CROSS-REFERENCE TO RELATED APPLICATION(S)

This application is a continuation-in-part of and claims the benefit of U.S. patent application Ser. No. 15/380,097, filed Dec. 15, 2016, which is a non-provisional application of U.S. Provisional Patent Application Ser. No. 62/267,366, filed Dec. 15, 2015, the entirety of each of which is hereby incorporated herein by reference.

This application claims the benefit of U.S. Provisional Patent Application Ser. No. 62/756,153, filed Nov. 6, 2018, the entirety of which is hereby incorporated herein by reference.

### BACKGROUND OF THE INVENTION

### 1. Field of the Invention

The present invention relates to neural interfaces and, more specifically, to a wireless neural interface system.

### 2. Description of the Related Art

Neural electrodes are designed to be implanted into the brains of patients to detect neural potentials generated as a result of neural activity. Such electrodes can be used to allow locked in individuals to control devices through a computer interface. In one use, neural electrodes have been used to generate phonemes as part of speech synthesis.

Neurotrophic electrodes are neural electrodes that include a neurotrophic factor that stimulates the growth of neurites into the neural electrode. One type neurotrophic electrode assembly includes one or more wires that extend into a glass cone. Neurites grown into the cone and an exposed portion of the wire (referred to as a “recording site”) collects data from the neurites. These electrode assemblies tend to be limited to having only one or two wires due to the bulkiness of the wires.

Many neural implants communicate with outside devices via cables that pass through the patient's scalp. Such cables require special care to prevent infection and can limit the patient's mobility.

Therefore, there is a need for a wireless system for communicating with neural implants.

### SUMMARY OF THE INVENTION

The disadvantages of the prior art are overcome by the present invention which, in one aspect, is a neural implant system for communicating neural impulses generated by a brain of a patient having a body. A neural implant electrode system is configured to be implanted in a selected site of the patient's brain. A wireless power receiver and communication circuit is in communication with the neural implant electrode and is configured to be disposed within the patient's body at a predetermined location. An external telemetry and power unit is configured to provide power to and to communicate with the wireless power receiver and communication circuit. The wireless power source and transceiver circuit is configured to operate outside of the patient's body.

In another aspect, the invention is a neural implant system for communicating neural impulses generated by a brain of a patient having a body. A neural implant electrode system is configured to be implanted in a selected site of the patient's brain. The neural implant system includes a neurotrophic electrode and an amplifier coupled thereto. A wireless power receiver and communication circuit is in communication with the neural implant electrode and is configured to be disposed within the patient's body at a predetermined location and to provide power to the neural implant electrode system and to receive a signal therefrom. The wireless power receiver and communication circuit is coupled to the neural implant electrode system via a biocompatible implantable cable. An external telemetry and power unit is configured to provide power to and to communicate with the wireless power receiver and communication circuit. The wireless power source and transceiver circuit is configured to operate outside of the patient's body.

These and other aspects of the invention will become apparent from the following description of the preferred embodiments taken in conjunction with the following drawings. As would be obvious to one skilled in the art, many variations and modifications of the invention may be effected without departing from the spirit and scope of the novel concepts of the disclosure.

### BRIEF DESCRIPTION OF THE FIGURES OF THE DRAWINGS

FIG. 1 is a perspective view of one embodiment of a neurotrophic electrode system.

FIG. 2 is a cut-away view of a second embodiment of a neurotrophic electrode system in which side openings are defined in a cone.

FIG. 3 is a schematic diagram of the embodiment shown in FIG. 2.

FIG. 4A is a schematic diagram of an embodiment of a multi-channel electrode assembly employing an undulating dielectric ribbon.

FIG. 4B is a narrow end view of a neurotrophic electrode employing the multi-channel electrode assembly shown in FIG. 4A.

FIG. 5A is a schematic diagram of an embodiment that includes a data acquisition and transmission module.

FIG. 5B is a schematic diagram of the embodiment shown in FIG. 5A, showing a detail of the data acquisition and transmission module.

FIG. 6 is a schematic diagram demonstrating the embodiment of FIG. 5A implanted in a brain.

FIG. 7 is a schematic diagram of one embodiment of a neural implant system employing wireless power transfer.

FIG. 8 is a schematic diagram of the system shown in FIG. 7 implanted in a patient.

### DETAILED DESCRIPTION OF THE INVENTION

A preferred embodiment of the invention is now described in detail. Referring to the drawings, like numbers indicate like parts throughout the views. Unless otherwise specifically indicated in the disclosure that follows, the drawings are not necessarily drawn to scale. The present disclosure should in no way be limited to the exemplary implementations and techniques illustrated in the drawings and described below. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.”

U.S. Pat. No. 4,852,573, issued to Kennedy, is incorporated herein by reference for the purpose of disclosing methods of making and using implantable neural electrodes. U.S. Pat. No. 9,124,125, issued to Leabman et al. discloses one embodiment of a wireless power receiver and communication circuit and is hereby incorporated by reference for the purpose of disclosing wireless power receiver and communication circuits.

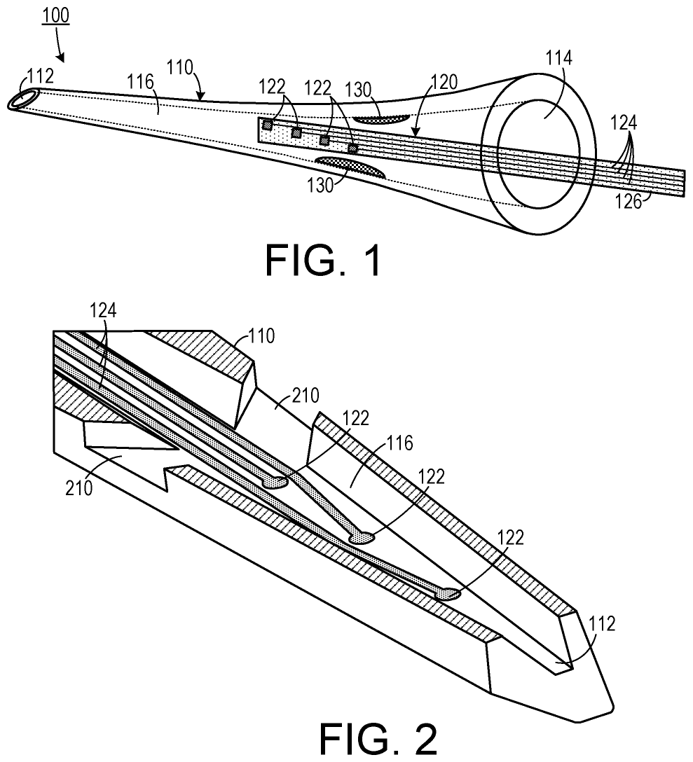

As shown in FIG. 1, one embodiment of a neurotrophic electrode 100 includes a cone 110 with an open small end 112 and an opposite open large end 114, which defines a cavity 116 therein. A multi-channel electrode assembly 120 is affixed to the cone 110 inside the cavity 116. The multi-channel electrode assembly 120 includes a plurality of exposed recording sites 122 that are each coupled to a different wire 124. In one embodiment, the recording sites 112 would be on the order of 10μ×10μ to 20μ×20μ. The wires 124 are encased in a dielectric ribbon 126 (such as a polyamide, polytetrafluoroethylene—which is sold under the mark Teflon®, or a poly(p-xylylene) polymer—which sold under the mark Parylene®, etc., film) and extend outside of the cone 110 through the open large end 114. Once implanted, neurons will grow into the cavity 116 through the opening in the small end 112, thereby securing the neurotrophic electrode 100.

The cone 110 can be made of such materials as glass, silicon, quartz, polyamide or one of many non-conducting materials that are stable in a neural environment. Typically, the wires 124 would be made of a non-corroding conductor such as platinum or gold. While the diagram shows only four wires/recordation sites, many more wire/recordation sites may be used. Using a large number of wire/recordation sites allows for the sensing of more complex neural potential patterns.

Prior to implantation, a material 130 that attracts neurites into the cone 110 is placed therein. Examples of such a material 130 include neural growth factors, nerve segments, endothelium, stem cells, and combinations thereof.

If stem cells are used, one method of acquiring such stem cells would be to take autologous stem cells a fat layer in the patient, which could be harvested subcutaneously one or two days before surgery using known methods. The stem cells would then be injected into the cone 110 shortly before implantation.

As shown in FIG. 2, in one embodiment can include side openings 210 to the cavity 116. Such openings 210 provide additional passages into which neurites can grow and also further secure the electrode in the neural tissue into which it is implanted.

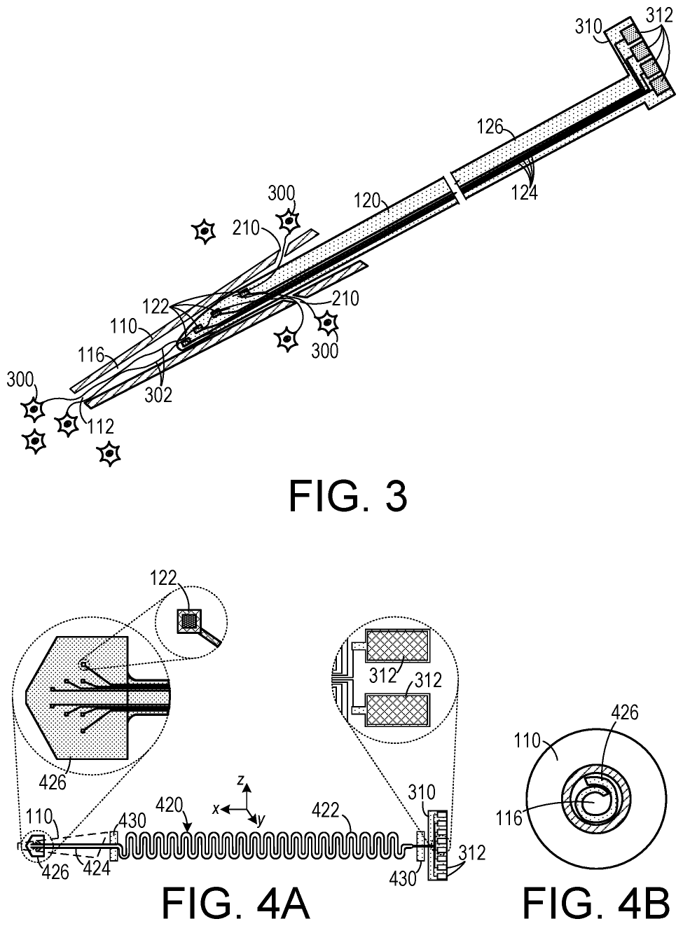

As shown in FIG. 3, one embodiment includes an elongated multi-channel electrode assembly 120 that terminates to a coupling surface 310 on which are exposed connection pads 312 for coupling the electrode to an external signal detection apparatus. While the wires 124 are embedded in the dielectric ribbon 126 or insulated with an applied resin, the recording sites 122 and the connection pads 312 are not insulated. The figure also shows neurites 302 extending from neurons 300 having grown in through passages 112 and 210 and in communication with the recording sites 122.

An alternate multi-channel electrode assembly 420 is shown in FIG. 4A, in which the dielectric ribbon 420 includes a straight portion 424 and an undulated ribbon portion 422 that extends outside of the cone 110. The undulated ribbon portion 422 is flexible in three axes that are orthogonal to each other (e.g., the x, y & z axes). A manipulation tab 430 can be attached to the ribbon 422 on either end (or on both ends) to provide a surface for holding and manipulating the electrode assembly during implantation. The straight portion 424 terminates in an end tab 426 on which the recording sites 122 are disposed. As shown in FIG. 4B, the end tab 426 is rolled up inside of the cavity 116 defined by the cone 110.

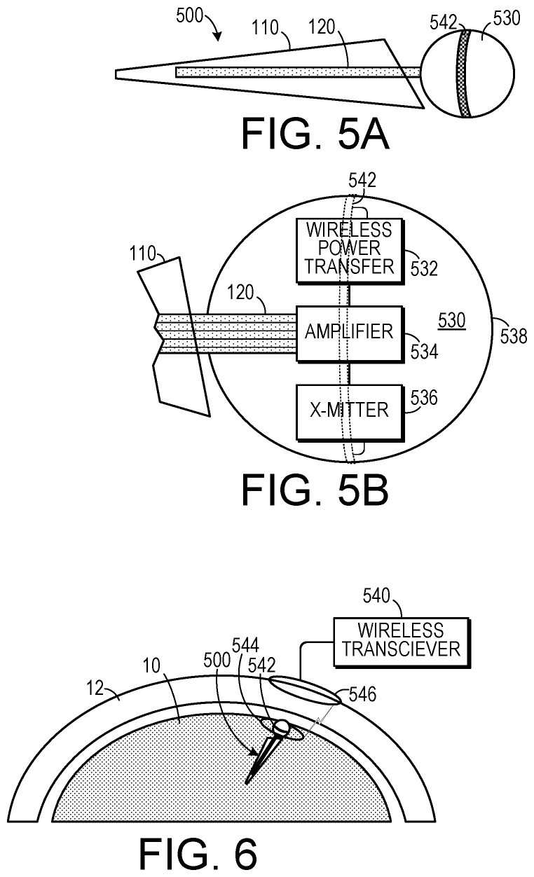

As shown in FIGS. 5A and 5B, one embodiment can be adapted for remote sensing in which the electrode assembly 500 is not physically coupled to a device that is external from the body. Such an assembly 500 includes a data acquisition and transmission module 530 that is coupled to the multi-channel electrode assembly 120. The data acquisition and transmission module 530 includes a wireless power transfer device 532 that receives a wireless signal from a remote device and that generates electrical power in response thereto. An amplifier 534, which is in data communication with the multi-channel electrode assembly 120, receives electrical power from the wireless power transfer device 532. The amplifier 534 amplifies data from the multi-channel electrode assembly 120 and communicates amplified data to a transmitter 536. The transmitter 536, which is powered by the wireless power transfer device 532, generates a wireless signal corresponding to amplified data. A bio-compatible casing 538 (such as a glass or plastic casing) envelops the data acquisition and transmission module 530. The bio-compatible casing 538 may be spaced apart from the second end of the cone 110 to accommodate tissue growth into the space there-between.

As shown in FIG. 6, after this embodiment is implanted in a brain 10, data acquired from the electrode system 500 can be acquired by a wireless transceiver device 540 without requiring wires passing through the skull 12. In this embodiment, a receiver coil 542 can be disposed about the periphery of the data acquisition and transmission module 530. A resonator coil 544 that has a resonant frequency that is common to a resonant frequency of the receiver coil 542 is disposed under the skull 12 about the data acquisition and transmission module 530. A transmitter coil 546, which has a resonant frequency in common with the resonator coil 544 and the receiver coil 542, is placed adjacent to the outside of the skull 12. Power is transferred to the electrode system 500 by applying a periodic signal to the transmitter coil 546, which causes it to resonate. This resonance induces a current in the resonator coil 544, which induces resonance therein. This resonance is inductively coupled to the receiver coil 542, which induces a current therein and causes power to be made available to the amplifier 534 and the transmitter 536. In collecting data, the process is essentially reversed: the transmitter 536 generates a signal onto which data from the amplifier 534 has been modulated. The signal is coupled to the receiver coil 542, inducing resonance that is inductively coupled to the resonator coil 544. This induces a resonating current in the resonator coil 544, which is inductively coupled to the transmitter coil 546. The signal induced in the transmitter coil 546 is detected and processed by the wireless transceiver device 540.

In other embodiments, the data acquisition and transmission module 530 could also include a memory module and a processor for more complex data processing. Also, the embodiment could be used for brain stimulation applications, in which the wireless transceiver 540 could be programmed to apply stimulating signals when certain neural potentials are sensed.

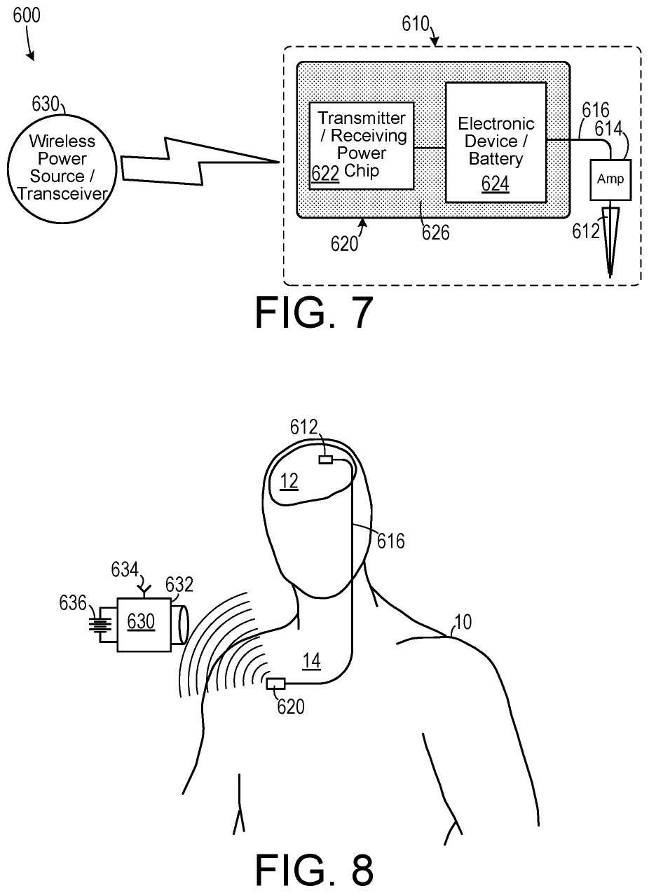

In one embodiment, as shown in FIGS. 7 and 8, a neural implant system 600 includes a neural implant electrode system 610 that receives power from and transmits data to an external telemetry and power unit 630 wirelessly. The data can be transmitted to a digital device (e.g., a computer) for use in controlling other devices (such as fans and lights) by locked-in patients, as well as in neurological research. The neural implant electrode system 610 includes a wireless power receiver and FM control module 620, which includes a data transmitter and a wireless power receiver 622, an electronic device 624 for conditioning a signal from the neural implant electrode system for transmission and a rechargeable battery for supplying power to the neural implant electrode system 610. The wireless power receiver and FM control module 620 is enveloped in a watertight biocompatible package 626.

In one embodiment, the wireless power receiver and communication circuit 620 includes at least one RF-to-DC rectifier and at least one antenna, in communication with the RF-to-DC rectifier for transmitting a power signal and for receiving a data signal. In one embodiment, the system can employ a wireless power induction and FM control module and it also receives data therefrom. A chipset employing one such unit (referred to as WattUp®) is available from Energous Corporation, 3590 N 1st Street, Suite 210, San Jose Calif., 95134.

The external telemetry and power unit 130 includes a power supply 636 and an RF power/data signal generator 632 that receives power from the power supply 636. One or more antennas 634 can be used to transmit power to and to receive data from the wireless power receiver and communication circuit 620.

The neural implant 612, which can be of a type disclosed above, is configured to be implanted into the neural tissue 12 of a patient 10 and is coupled to a phase 1 amplifier 614, which could be mounted on the patient's skull. A bio-compatible implantable cable 616 (such as a Medtronic® cable) couples the phase 1 amplifier 614 to a wireless power induction and FM control module 620 and is implanted in the patient at a predetermined location (e.g., the patient's chest 14 or behind one of the patient's ears). Neural impulses generated in the patient's 10 brain 12 are sensed by the neural implant 612, amplified by the amplifier 114 and transmitted by the communication circuit 620.

In one embodiment, an external telemetry and power unit could be magnetically fixed to the scalp and it would receive the wireless power and inductively couple to the coil under the scalp.

In one embodiment the device picks up wireless power from the external telemetry and power unit from up to 30 feet away. One embodiment includes an implantable amplifier to record the neural signals. It has a power induction system and FM transmitter as well a control chip. The amplifier is mounted on the skull and the remaining parts of the system are mounted on the chest wall connected by a wire lead. It is on the electronic connection between the electrodes in the brain and the external world. In one embodiment, the amplifiers are on the skull under the scalp and a connection would lead to the chest wall that contains the power induction system, control system and FM transmitter.

Although specific advantages have been enumerated above, various embodiments may include some, none, or all of the enumerated advantages. Other technical advantages may become readily apparent to one of ordinary skill in the art after review of the following figures and description. It is understood that, although exemplary embodiments are illustrated in the figures and described below, the principles of the present disclosure may be implemented using any number of techniques, whether currently known or not. Modifications, additions, or omissions may be made to the systems, apparatuses, and methods described herein without departing from the scope of the invention. The components of the systems and apparatuses may be integrated or separated. The operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set. It is intended that the claims and claim elements recited below do not invoke 35 U.S.C. § 112(f) unless the words “means for” or “step for” are explicitly used in the particular claim. The above described embodiments, while including the preferred embodiment and the best mode of the invention known to the inventor at the time of filing, are given as illustrative examples only. It will be readily appreciated that many deviations may be made from the specific embodiments disclosed in this specification without departing from the spirit and scope of the invention. Accordingly, the scope of the invention is to be determined by the claims below rather than being limited to the specifically described embodiments above.

What is claimed is:

1. A neural implant system for communicating neural impulses generated by a brain of a patient having a body, comprising:
(a) a neural implant electrode system configured to be implanted in a selected site of the patient's brain;
(b) a wireless power receiver and communication circuit in communication with the neural implant electrode, configured to be disposed within the patient's body at a predetermined location;
(c) an external telemetry and power unit configured to provide power to and to communicate with the wireless power receiver and communication circuit, the wireless power source and transceiver circuit configured to operate outside of the patient's body.

2. The neural implant system of claim 1, wherein the neural implant electrode system comprises a neurotrophic electrode and an amplifier coupled thereto, the amplifier configured to receive power from the wireless power receiver and communication circuit.

3. The neural implant system of claim 2, wherein the neurotrophic electrode comprises:
(a) a non-conductive cone that consists essentially of a material that is stable in a neural environment and that defines a cavity, the cavity opens to a small opening at a first end of the cone and opens to a large opening at a second end of the cone that is opposite the first end;
(b) an electrode assembly including at least one recording site that is disposed within the cavity defined by the cone, the recording site coupled to a wire that extends out of the large end of the cone; and
(c) a neurite-attracting substance disposed within the cone.

4. The neurotrophic electrode system of claim 3, wherein the non-conductive cone comprises a material selected from a list consisting of: glass, quartz, silicon, and polyamide.

5. The neurotrophic electrode system of claim 3, wherein the neurite-attracting substance comprises a substance selected from a list consisting of: neural growth factors, nerve segments, endothelium, stem cells, and combinations thereof.

6. The neurotrophic electrode system of claim 3, wherein the wire comprises a selected one of platinum or gold.

7. The neural implant system of claim 1, wherein the wireless power receiver and communication circuit is coupled to the neural implant electrode system via a biocompatible implantable cable.

8. The neural implant system of claim 1, wherein the predetermined location comprises the patient's chest.

9. The neural implant system of claim 1, wireless power receiver and communication circuit wherein the predetermined location comprises a location behind an ear of the patient.

10. The neural implant system of claim 1, wireless power receiver and communication circuit includes:
(a) a circuit that includes a data transmitter and a wireless power receiver;
(b) an electronic device for conditioning a signal from the neural implant electrode system for transmission; and
(c) a rechargeable battery.

11. The neural implant system of claim 10, wherein the wireless power receiver and communication circuit is enveloped in a watertight biocompatible package.

12. The neural implant system of claim 1, wireless power receiver and communication circuit includes:
(a) at least one RF-to-DC rectifier; and
(b) at least one antenna, in communication with the RF-to-DC rectifier for transmitting a power signal and for receiving a data signal.

13. The neural implant system of claim 1, external telemetry and power unit comprises
(a) a power supply; and
(b) an RF power/data signal generator, coupled to the power supply.

14. A neural implant system for communicating neural impulses generated by a brain of a patient having a body, comprising:
(a) a neural implant electrode system configured to be implanted in a selected site of the patient's brain, the neural implant system including a neurotrophic electrode and an amplifier coupled thereto;
(b) a wireless power receiver and communication circuit in communication with the neural implant electrode, configured to be disposed within the patient's body at a predetermined location and to provide power to the neural implant electrode system and to receive a signal therefrom, the wireless power receiver and communication circuit being coupled to the neural implant electrode system via a biocompatible implantable cable;
(c) an external telemetry and power unit configured to provide power to and to communicate with the wireless power receiver and communication circuit, the wireless power source and transceiver circuit configured to operate outside of the patient's body.

15. The neural implant system of claim 14, wherein the neurotrophic electrode comprises:
(a) a non-conductive cone that consists essentially of a material that is stable in a neural environment and that defines a cavity, the cavity opens to a small opening at a first end of the cone and opens to a large opening at a second end of the cone that is opposite the first end;
(b) an electrode assembly including at least one recording site that is disposed within the cavity defined by the cone, the recording site coupled to a wire that extends out of the large end of the cone; and
(c) a neurite-attracting substance disposed within the cone.

16. The neural implant system of claim 14, wherein the predetermined location comprises a selected on of the patient's chest or a location behind an ear of the patient.

17. The neural implant system of claim 14, wireless power receiver and communication circuit includes:
(a) a circuit that includes a data transmitter and a wireless power receiver;
(b) an electronic device for conditioning a signal from the neural implant electrode system for transmission; and
(c) a rechargeable battery.

18. The neural implant system of claim 17, wherein the wireless power receiver and communication circuit is enveloped in a watertight biocompatible package.

19. The neural implant system of claim 14, wireless power receiver and communication circuit includes:
(a) at least one RF-to-DC rectifier; and
(b) at least one antenna, in communication with the RF-to-DC rectifier for transmitting a power signal and for receiving a data signal.

20. The neural implant system of claim 14, external telemetry and power unit comprises
(a) a power supply; and
(b) an RF power/data signal generator, coupled to the power supply.
